(19) United States
(12) Patent Application Publication
(10) Pub. No.: US 2024/0066230 A1
(43) Pub. Date: Feb. 29, 2024

(54) FREEZE-DRY POWDER INJECTOR RESTORING DEVICE

(71) Applicant: CHANG-TZU CHEN, Taipei City (TW)

(72) Inventors: CHANG-TZU CHEN, Taipei City (TW); MING-SHEN WANG, New Taipei City (TW); TE-LING KO, Taoyuan City (TW)

(21) Appl. No.: 17/895,974

(22) Filed: Aug. 25, 2022

(51) Int. Cl. A61M 5/31; A61M 5/315

(52) U.S. Cl. CPC A61M 5/3145; A61M 5/31596

(57) ABSTRACT

A freeze-dry powder injector restoring device has a cylinder, a filtering unit, and a bottom cover. The cylinder has a connecting segment having an upper opening and a cylinder body. The cylinder body and the connecting segment are respectively located in two ends of the cylinder. The cylinder body has an inner space and a lower opening connecting the inner space and the upper opening. The filtering unit is mounted in the cylinder and divides the inner space into multiple rooms. The bottom cover has a tube segment and a flange segment protruding from an outer surface of the tube segment and surrounding the tube segment. The bottom cover covers and abuts the lower opening of the cylinder via the flange segment. The freeze-dry powder accommodated in the cylinder can be easily mixed with the solvent to be restored.

### BACKGROUND OF THE INVENTION

### 1. Field of the Invention

The present invention relates to a medical device, especially to a freeze-dry powder injector restoring device that can contain and restore freeze-dried medicine.

### 2. Description of the Prior Arts

Recently, the US Food and Drug Administration (FDA) has being paying more and more attention to the influence of human factors in the process of drug reconstitution and injection when approving new drugs or preparations. If a pharmaceutical company fails to provide data demonstrating sufficient reliability during drug reconstitution and injection, the approval will not be established. Due to the lack of stability of supplying biologics in liquid form at room temperature, refrigerated transport and storage are required to maintain stability of biologics throughout the life of the product. However, the efficacy of some liquid-form biologics still might be decreased over time.

In order to ensure the stability and efficacy of the drug, there is a process that freezes the liquid medicine into a solid state in a sterile environment, and sublimates and dries the water after vacuuming to form a sterile powder injection, and then mixes the raw medicine with some excipients or dissolves it in some solvent. After certain processing, the liquid medicine is made into different forms of preparations, the so-called freeze-dry powder injector. The freeze-dry powder injector has many advantages such as no denaturation, small loss of volatile components and long-term storage. When injection is required, the restoration step of the freeze-dry powder injector is performed, that is, the solvent is added and the freeze-dry powder injector is fused into liquid form. Then, the restored medicine can be drawn by an injector for the following injection.

However, a conventional process for restoring the freeze-dry powder injector is complicated, including a dozen steps and involving an injector, a single-dose or multi-dose vial containing medicine, a vial or injector with diluent, a blunt needle, and a narrow-bore needle for injection. For a medical worker who may perform a large-scale vaccination with dozens to hundreds of injections per day, the restoration of the freeze-dry powder injector is troublesome and time-consuming, and there will also be a certain number of mistakes in the proportioning of doses and errors in operation during the process of restoration. This increases the risk of needle sticking injuries for medical workers and reduces the willingness of medical workers to do so, which has been a topic of concern for a long time. In addition, the lack of an efficient restoration system also hinders commercialization.

Nowadays, a double chamber restoring injector is provided in the medical device field. The double chamber restoring injector has two chambers. One of the chambers is used to contain a freeze-dry powder injector while the other one is used to contain a solvent. The two chambers are isolated from each other by a rubber stopper, so that the freeze-dry powder injector and the solvent are isolated before injection. When injecting, a user presses a plunger of the injector to make the solvent flow through a passage between the rubber stopper and an inner wall of the injector and flow into the chamber containing the freeze-dry powder injector to be mixed with the freeze-dry powder injector.

However, the process of pre-filling the freeze-dry powder injector and solvent at the same time is complicated, and the weight of the solvent will therefore be added during transportation, and the solvent also has a shorter shelf life, which overall offset the advantages of the freeze-dry powder injector in transportation weight and shelf life. Therefore, the above problems must be solved.

To overcome the shortcomings, the present invention provides a freeze-dry powder injector restoring device to mitigate or obviate the aforementioned problems.

### SUMMARY OF THE INVENTION

The main objective of the present invention is to provide a freeze-dry powder injector restoring device that has a filtering unit and a bottom cover mounted in a cylinder, so that the freeze-dry powder accommodated in the cylinder can be easily mixed with the solvent to be restored.

The freeze-dry powder injector restoring device has a cylinder, a filtering unit, and a bottom cover. The cylinder has a connecting segment and a cylinder body. The connecting segment has an upper opening. The cylinder body and the connecting segment are respectively located in two ends of the cylinder. The cylinder body has an inner space and a lower opening. The lower opening is connected to the inner space and the upper opening. The filtering unit is mounted in the cylinder and divides the inner space into multiple rooms. The bottom cover has a tube segment and a flange segment. The flange segment protrudes from an outer surface of the tube segment and surrounding the tube segment. The bottom cover covers and abuts the lower opening of the cylinder via the flange segment.

Since the freeze-dry powder is pre-stored in the third chamber of the cylinder, and the cylinder can be directly combined with the injection needle and the injector, the portability of the medication in freeze-dry powder form can be improved for medical personnel or self-injecting patients, and the restoring process becomes efficient and easy by reducing the process, steps, time, and labor required for restoring and dispensing an injection dose, thereby helping minimizing the risk of administration errors. For pharmaceutical manufacturers, compared with a conventional restoring appliance set, the freeze-dry powder injector restoring device of the present invention is capable of containing freeze-dry powder, transforming liquid medicine into freeze-dry powder, and restoring freeze-dry powder by only the cylinder, the filtering unit, and the bottom cover, which helps reducing the cost on transportation and packaging.

Other objectives, advantages and novel features of the invention will become more apparent from the following detailed description when taken in conjunction with the accompanying drawings.

### BRIEF DESCRIPTION OF THE DRAWINGS

FIG. 1 is a perspective view of a freeze-dry powder injector restoring device in accordance with the present invention;

FIG. 2 is an exploded view of the freeze-dry powder injector restoring device in FIG. 1;

FIG. 3 is another exploded view of the freeze-dry powder injector restoring device in FIG. 1, shown in another direction;

FIG. 4 is a side view in cross-section of the freeze-dry powder injector restoring device in FIG. 1, showing the filtering unit;

FIG. 5 is another side view in cross-section of the freeze-dry powder injector restoring device in FIG. 1;

FIG. 6 is an operational view of the freeze-dry powder injector restoring device in FIG. 1, showing a filling process of liquid medicine;

FIG. 7 is another operational view of the freeze-dry powder injector restoring device in FIG. 1, showing an exhausting process of air;

FIG. 8 is still another side view in cross-section of the freeze-dry powder injector restoring device in FIG. 1, showing the third chamber containing the freeze-dry powder;

FIG. 9 is still another operational view of the freeze-dry powder injector restoring device in FIG. 1, showing the freeze-dry powder restored into liquid medicine; and

FIG. 10 is still another side view in cross-section of the freeze-dry powder injector restoring device in FIG. 1, showing the freeze-dry powder injector restoring device assembled with an injection needle and an injector.

### DETAILED DESCRIPTION OF THE PREFERRED EMBODIMENTS

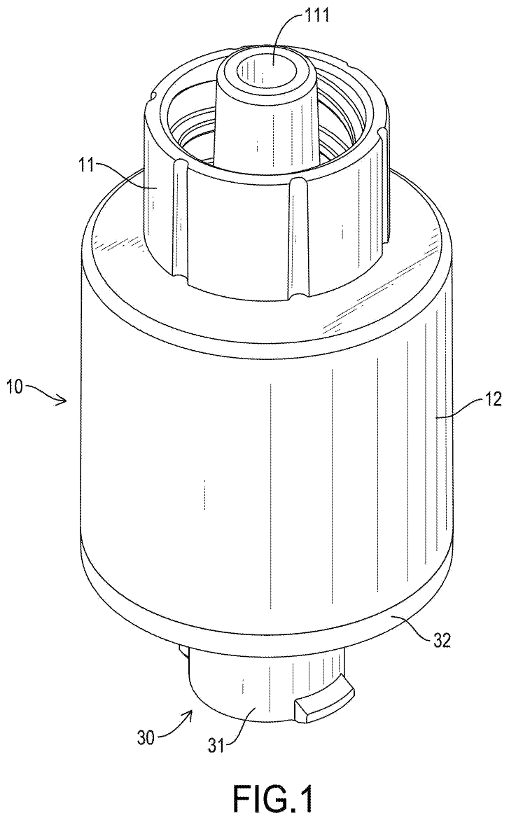

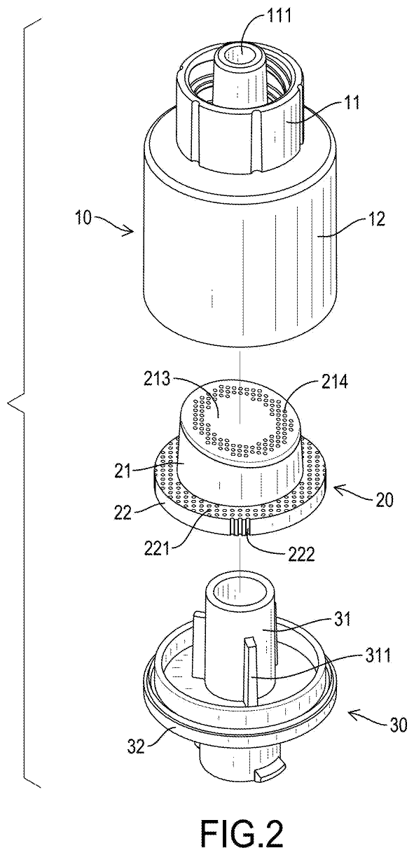

With reference to FIG. 1 and FIG. 2, the freeze-dry powder injector restoring device of the present invention includes a cylinder 10, a filtering unit 20 and a bottom cover 30.

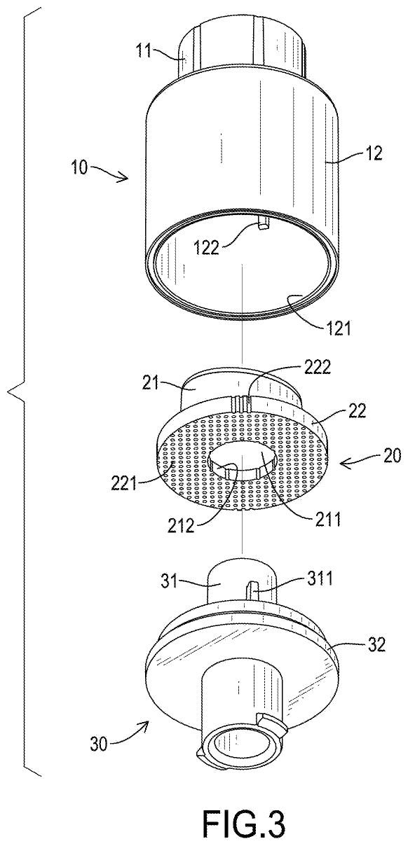

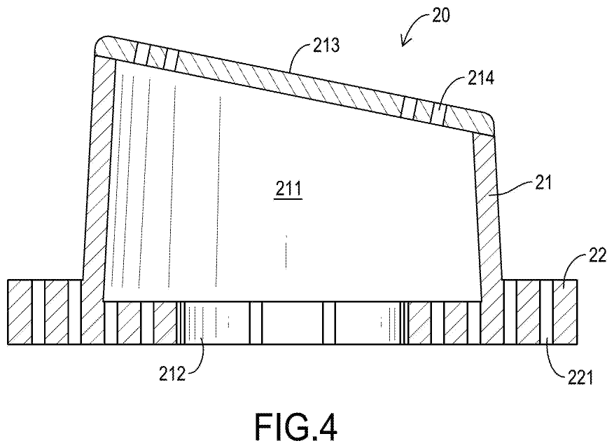

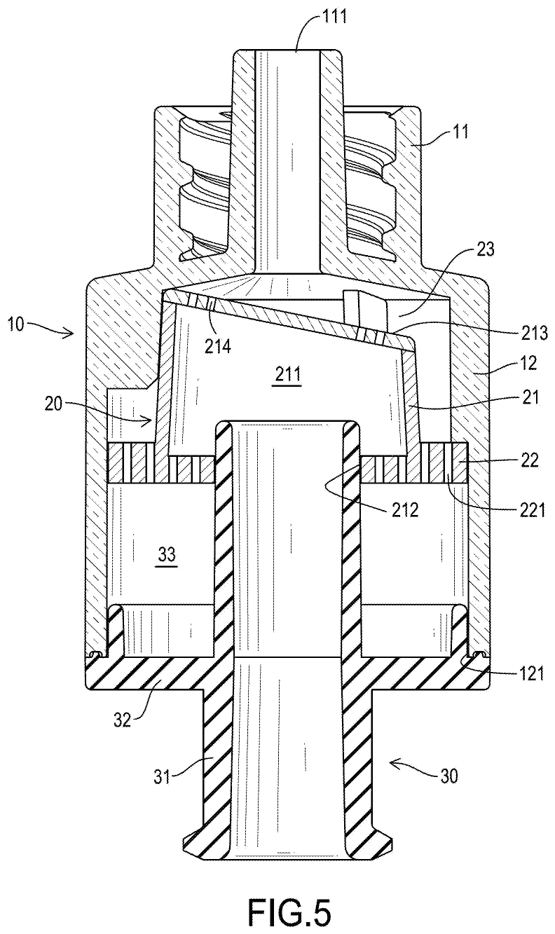

With reference to FIG. 2 to FIG. 5, the cylinder 10 has a connecting segment 11 and a cylinder body 12. The connecting segment 11 and the cylinder body 12 are respectively located in two ends of the cylinder 10. The connecting segment 11 has an upper opening 111. The connecting segment 11 is preferably a Luer taper. The cylinder body 12 is cylindrical in shape and has an inner space and a lower opening 121 connected to the inner space and the upper opening 111. Multiple positioning ribs 122 are formed on an inner surface of the cylinder body, extend along an axial direction of the cylinder body, and are spaced apart from each other. In this embodiment, the cylinder 10 is made of a transparent material, but the configuration of the cylinder 10 is not limited thereto, as the cylinder 10 can be in any configuration as needed.

The filtering unit 20 has a top filtering segment 21 and a flange filtering segment 22. The top filtering segment 21 forms a first chamber 211 and a first opening 212. The first opening 212 and the first chamber 211 are connected to each other. The top filtering segment 21 has a top surface 213. The top surface 213 is an inclined surface and has multiple top filtering openings 214 formed therethrough. The top surface 213 is inclined from an end to another end thereof. The top filtering openings 214 are arranged in a circle and are spaced apart from each other. The flange filtering segment 22 protrudes from an outer surface of the top filtering segment 21, surrounds the top filtering segment 21, and is adjacent to the first opening 212. The flange filtering segment 22 forms multiple flange filtering openings 221 and multiple flange positioning grooves 222. The flange filtering openings 221 are formed through the flange filtering segment 22. The flange positioning grooves 222 are concave from an outer annular surface of the flange filtering segment 22 and respectively correspond in positions to the positioning ribs 122. The filtering unit 20 is mounted in the inner space of the cylinder 10 and the top surface 213 is adjacent to the connecting segment 11. The positioning ribs 122 abut the flange positioning groove 222. The filtering unit 20 and the cylinder 10 surround and form a second chamber 23. In this embodiment, the filtering unit 20 is made of a transparent material. The top filtering segment 21 is breathable, impermeable, not hygroscopic, and hydrophobic. The flange filtering segment 22 is breathable, permeable, not hygroscopic, and dustproof. But the configuration of the filtering unit 20 is not limited thereto, and is not limited to holes or gaps, as the filtering unit 20 can be configured as needed and can be made of other materials or made in other structures that have the same filtering effect. In other embodiments, the top filtering segment 21 and the flange filtering segment 22 can also be two independent parts, and the filtering unit 20 can also be made of woven materials that have multiple pores.

The bottom cover 30 has a tube segment 31 and a flange segment 32. The flange segment 32 is an annular body protruding from an outer surface of the tube segment 31 and surrounding the tube segment 31. Multiple rib sheets 311 protrude from the outer surface of the tube segment 31, are spaced apart from each other, and are adjacent to an end of the tube segment 31. Each of the rib sheets 311 extends along an axial direction of the tube segment 31. Another end of the tube segment 31 is a Luer interface. The bottom cover 30 covers the lower opening 121 of the cylinder 10 via the flange segment 32. An end of the tube segment 31 is mounted in the first opening 212 and is connected to the first chamber 211. An end surface of each of the rib sheets 311 abuts another side surface of the filtering unit 20. The bottom cover 30 and the filtering unit 20 surround and form a third chamber 33. In this embodiment, the bottom cover 30 is made of, but not limited to, a transparent material.

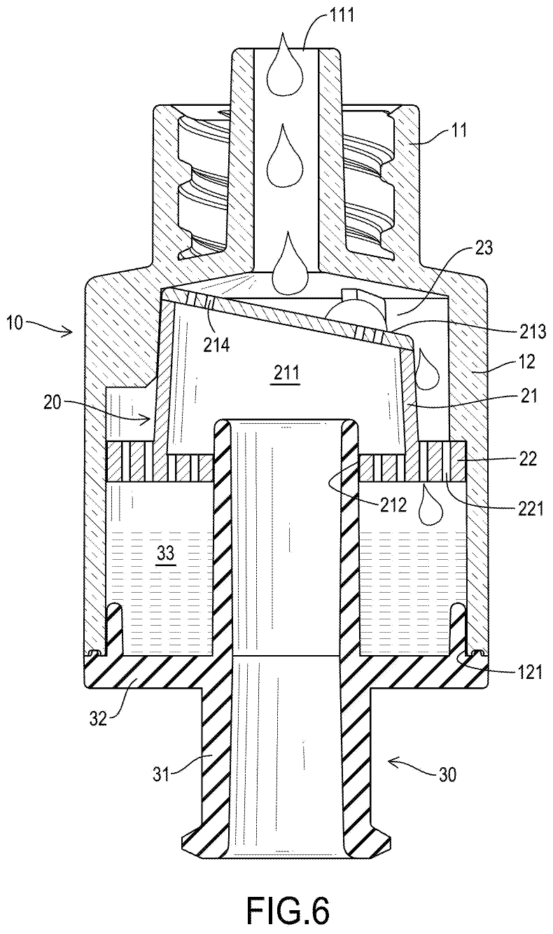

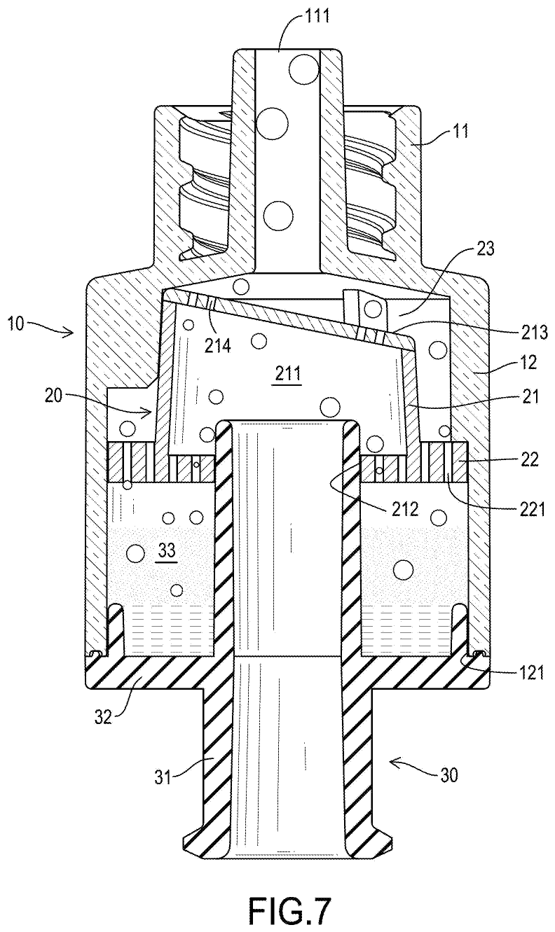

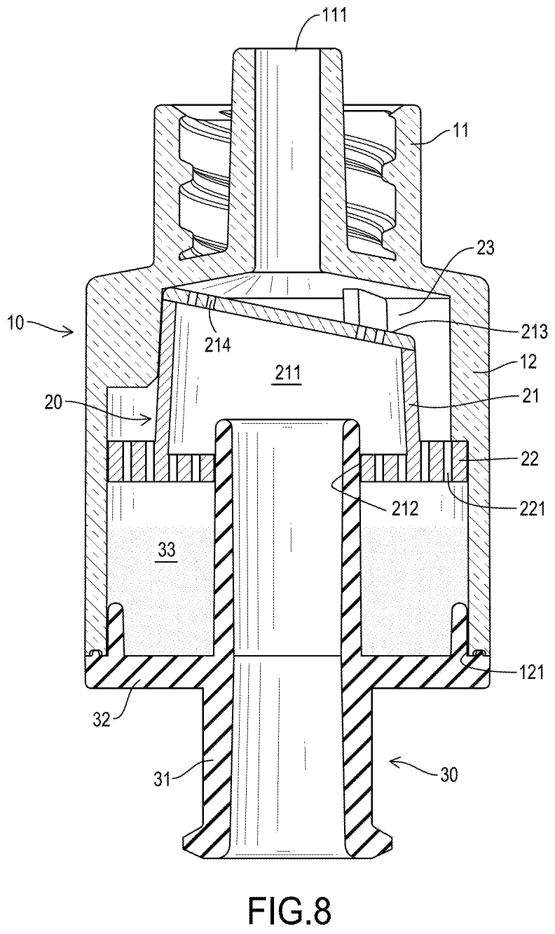

When filling the liquid medicine into the present invention, with reference to FIG. 6, an operator fills the liquid medicine via the upper opening 111 of the cylinder 10 into the second chamber 23. When the liquid medicine passes the top surface 213 of the filtering unit 20, since the top surface 213 is inclined, the top surface 213 guides the liquid medicine to flow to the flange filtering segment 22 and then to flow into the third chamber 33 through the flange filtering openings 221. With reference to FIG. 7, the liquid medicine is transformed into freeze-dry powder by aseptic freeze drying process. With reference to FIG. 8, the filtering unit 20 prevents the liquid medicine from dripping into the tube segment 31 and prevents expelling sublimation water vapor. The flange filtering segment 22 provides exhaustion function and prevents the freeze-dry powder from spreading out of the third chamber 33. The present invention is packaged by an aluminum foil vacuum packaging (not shown in drawings).

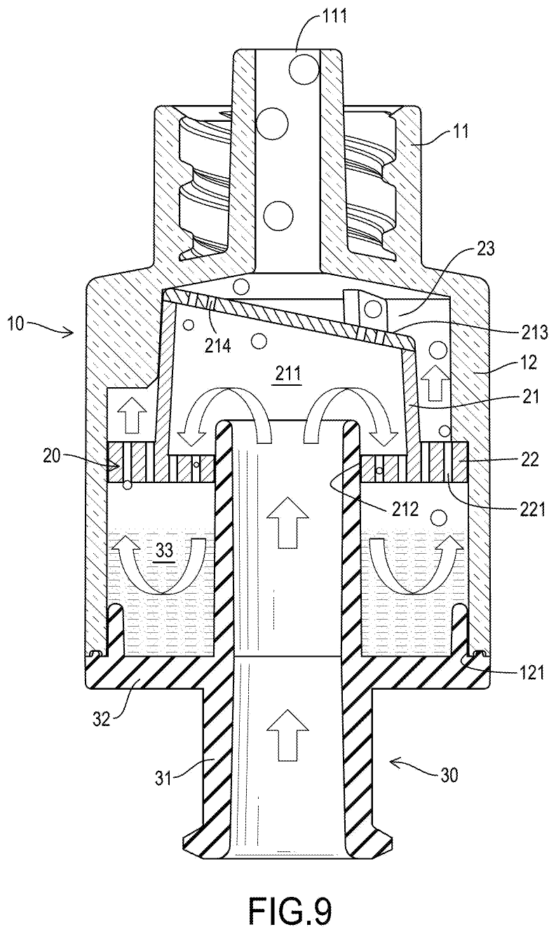

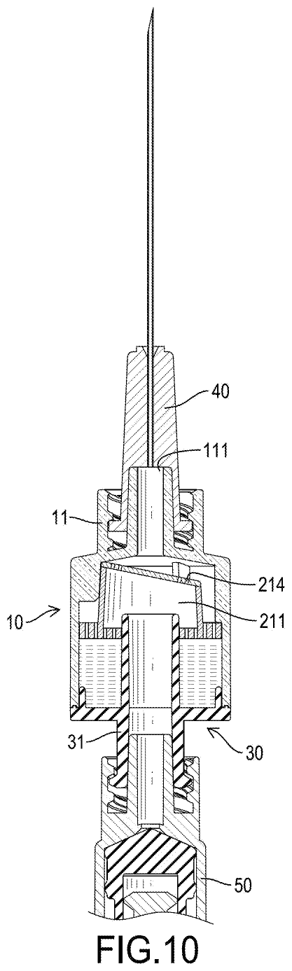

With reference to FIGS. 9 and 10, when in use, the present invention is operated with an injection needle 40 and an injector 50. The injection needle 40 is sleeved on the connecting segment 11 of the cylinder 10. The injection needle 40 can be an Eclipse Needle with Syringe. The injector 50 is mounted in the other end of the tube segment 31 of the bottom cover 30. The connections of the injection needle 40 and the injector 50 are conventional and will not be described in detail. The freeze-dry powder 60 is contained in the third chamber 33. When restoring the freeze-dry powder 60 to the liquid medicine, assuming that some of the flange filtering openings 221 are blocked by the freeze-dry powder 60, the operator must feed the solvent (such as saline) from the injector 50 into the first chamber 211, and during feeding the air in the first chamber 211 must be exhausted from the top filtering opening 214 via the upper opening 111. After the solvent is fed into the first chamber 211, the solvent first contacts and restores the freeze-dry powder 60 stuck in the flange filtering opening 221, and then passes through the flange filtering opening 221 by the small pressure continuously input from the injector 50, and the solvent keeps dissolving and restoring the freeze-dry powder 60 in the third chamber 33 into the liquid medicine thereafter. The liquid medicine will then pass through the flange filtering openings 221 near outer side into the second chamber 23, and the restoring process is accomplished. After the restoring process, the medical personnel is allowed to simply push the liquid medicine to the injection needle 40 via the upper opening 111 during injection.

In the aforementioned process, since the freeze-dry powder 60 is pre-stored in the third chamber 33 of the cylinder 10, and the cylinder 10 can be directly combined with the injection needle 40 and the injector 50, the portability of the medication in freeze-dry powder form can be improved for medical personnel or self-injecting patients, and the restoring process becomes efficient and easy by reducing the process, steps, time, and labor required for restoring and dispensing an injection dose, thereby helping minimizing the risk of administration errors. For pharmaceutical manufacturers, compared with a conventional restoring appliance set, the freeze-dry powder injector restoring device in the present invention is capable of containing freeze-dry powder, transforming liquid medicine into freeze-dry powder, and restoring freeze-dry powder by only the cylinder 10, the filtering unit 20, and the bottom cover 30, which helps reducing the cost on transportation and packaging.

In the aforementioned process, since the upper opening 111 and the tube segment 31 are both not sealed with a plug, the air in the first chamber 211, the second chamber 23, and the third chamber 33 can be easily exhausted when vacuum packaging, so that the freeze drying process and the vacuum packaging process can both be accomplished under sterile environment.

In the aforementioned process, both the connecting segment 11 and the tube segment 31 have compatibility and are adapted for ISO standard Luer socket and standard screw connecting hole. The connecting segment 11 can be installed with the injection needle, and the connecting segment 11 and the tube segment 31 can be directly connected with the injector or the drip tube.

In the aforementioned process, since the cylinder 10, the filtering unit and the bottom cover 30 are made of transparent materials, users can 16 directly see the freeze drying and restoring status of the internal medicine.

In the aforementioned process, the filtering unit 20 prevents the injection needle 40 from being blocked by the freeze-dry powder 60 during the restoring process.

In the aforementioned process, since the present invention can be used as a container for freeze drying and restoring process after cleaning and sterilization, and the processes are accomplished under sterile environment, and there is no pharmacy bottle change and no contact of other tools during the processes, the chance of drug contamination is greatly reduced.

In the aforementioned process, the present invention can be used to accomplish the restoring process without being assembled with the injection needle 40. In other words, the present invention has non-needle-restoring function for the preparation of a freeze-dry powder injector.

In the aforementioned process, when a second medicine can be used as the solvent for the freeze-dry powder injector and the medicine needs to be mixed, the present invention can simultaneously perform the restoring process of freeze-dry powder and the use of mixed medicaments.

In the aforementioned process, since the top surface 213 of the top filtering segment 21 is inclined, the liquid medicine can be guided to flow downward and the air can be exhausted, thereby solving the stuck bubble issue in the corner.

Even though numerous characteristics and advantages of the present invention have been set forth in the foregoing description, together with details of the structure and features of the invention, the disclosure is illustrative only. Changes may be made in the details, especially in matters of shape, size, and arrangement of parts within the principles of the invention to the full extent indicated by the broad general meaning of the terms in which the appended claims are expressed.

What is claimed is:

1. A freeze-dry powder injector restoring device comprising:
a cylinder having
a connecting segment having
an upper opening; and
a cylinder body; the cylinder body and the connecting segment respectively located in two ends of the cylinder; the cylinder body having
an inner space; and
a lower opening connected to the inner space and the upper opening;
a filtering unit mounted in the cylinder and dividing the inner space into multiple rooms; and
a bottom cover having
a tube segment; and
a flange segment protruding from an outer surface of the tube segment and surrounding the tube segment; the bottom cover covering and abutting the lower opening of the cylinder via the flange segment.

2. The freeze-dry powder injector restoring device as claimed in claim 1, wherein the filtering unit has
a top filtering segment having
multiple top filtering openings formed therethrough; and
a flange filtering segment having
multiple flange filtering openings formed therethrough; the top filtering segment and the flange filtering segment surrounding and forming a first chamber.

3. The freeze-dry powder injector restoring device as claimed in claim 2, wherein
the filtering unit has
a first opening formed therethrough and connected to the first chamber; the tube segment mounted in the first opening and connected to the first chamber; and
the top filtering segment has
a top surface adjacent to the connecting segment; each of the top filtering openings located on the top surface; the flange filtering segment protruding from an outer surface of the top filtering segment, surrounding the top filtering segment, and being adjacent to the first opening.

4. The freeze-dry powder injector restoring device as claimed in claim 2, wherein
multiple positioning ribs are formed on an inner surface of the cylinder body, extend along an axial direction of the cylinder body, and are spaced apart from each other; and
multiple flange positioning grooves are formed on the filtering unit and respectively abut the positioning ribs.

5. The freeze-dry powder injector restoring device as claimed in claim 3, wherein
multiple positioning ribs are formed on an inner surface of the cylinder body, extend along an axial direction of the cylinder body, and are spaced apart from each other; and
multiple flange positioning grooves are formed on the filtering unit and respectively abut the positioning ribs.

6. The freeze-dry powder injector restoring device as claimed in claim 4, wherein
each of the flange positioning grooves is concave from an outer annular surface of the flange filtering segment and extends along the axial direction.

7. The freeze-dry powder injector restoring device as claimed in claim 5, wherein
each of the flange positioning grooves is concave from an outer annular surface of the flange filtering segment and extends along the axial direction.

8. The freeze-dry powder injector restoring device as claimed in claim 6, wherein the top surface of the top filtering segment is an inclined surface sloping downward from an end to another end thereof.

9. The freeze-dry powder injector restoring device as claimed in claim 7, wherein the top surface of the top filtering segment is an inclined surface sloping downward from an end to another end thereof.

10. The freeze-dry powder injector restoring device as claimed in claim 8, wherein the top filtering openings are arranged annularly and are spaced apart from each other.

11. The freeze-dry powder injector restoring device as claimed in claim 9, wherein the top filtering openings are arranged annularly and are spaced apart from each other.

12. The freeze-dry powder injector restoring device as claimed in claim 10, wherein
multiple rib sheets protrude from the outer surface of the tube segment, are adjacent to an end of the tube segment, and are spaced apart from each other; each of the rib sheets extends along the axial direction; an end surface of each of the rib sheets abuts a side surface of the flange filtering segment.

13. The freeze-dry powder injector restoring device as claimed in claim 11, wherein
multiple rib sheets protrude from the outer surface of the tube segment, are adjacent to an end of the tube segment, and are spaced apart from each other; each of the rib sheets extends along the axial direction; an end surface of each of the rib sheets abuts a side surface of the flange filtering segment.
